(19) United States
(12) Patent Application Publication
(10) Pub. No.: US 2023/0207077 A1
(43) Pub. Date: Jun. 29, 2023

(54) COOPERATION SERVER, SYSTEM, IMMUNE CERTIFICATE GENERATION METHOD, AND NON-TRANSITORY COMPUTER-READABLE MEDIUM

(71) Applicant: NEC Corporation, Minato-ku, Tokyo (JP)

(72) Inventors: Michio KAJI, Tokyo (JP); Akihiko Ichikawa, Tokyo (JP); Masanori Kobayashi, Tokyo (JP); Taizo Higuchi, Tokyo (JP); Takeshi Yamaguchi, Tokyo (JP)

(73) Assignee: NEC Corporation, Minato-ku, Tokyo (JP)

(21) Appl. No.: 17/927,047

(22) PCT Filed: May 28, 2020
(86) PCT No.: PCT/JP2020/021119

§ 371 Date: Nov. 22, 2022

(51) Int. Cl. G16H 10/60; G16H 70/00

(52) U.S. Cl. CPC G16H 10/60; G16H 70/00

(57) ABSTRACT

A cooperation server (100) verifying that a user is not suffering from an infectious disease includes a reception unit (101), a transmission unit (102), and a generation unit (103). The reception unit (101) receives a request for generating an immune certificate for an infectious disease. The transmission unit (102) transmits an identity verification request regarding an applicant who requests generation of the immune certificate, to an authentication server storing information regarding each of a plurality of users whose identities are confirmed. The generation unit (103) transmits, to a public institution server, personal identification information being information uniquely defining the applicant, identification information issued by a public institution, and acquired at a time of responding to the identity verification request thereby acquiring infectious disease response information regarding an applicant of the personal identification information. The generation unit (103) generates an immune certificate, based on the acquired infectious disease response information.

### TECHNICAL FIELD

The present disclosure relates to a cooperation server, a system, an immune certificate generation method, and a non-transitory computer-readable medium.

### BACKGROUND ART

In recent years, various services using biometric information have begun to be popularized. For example, face authentication is used for various procedures (check-in, baggage deposit, etc.), which are performed in an airport, check-in of a hotel, and the like.

In the service using the face authentication, processing is performed in the following flow. First, a terminal (a terminal installed in an airport or a hotel) acquires a face image of a user, and generates a feature amount (feature vector) characterizing the face image. The generated feature amount is transmitted to a server on a network.

The server includes a database storing biometric information and personal information (name, address, etc.) of a user who receives a service by the face authentication. When the server acquires a collation request from the terminal, the server searches (collates) the database and specifies biometric information and personal information being associated to the collation request from the terminal. The server transmits the specified personal information to the terminal, and the terminal installed in the airport or the like conducts an operation based on the acquired personal information.

For example, Patent Literature 1 discloses a private accommodation management server that performs identification confirmation of a guest by using a personal video captured by a mobile terminal and unlocks a key of a room in a private accommodation service system.

### CITATION LIST

### Patent Literature

[Patent Literature 1] Japanese Unexamined Patent Application Publication No. 2018-101235

### SUMMARY OF INVENTION

### Technical Problem

There is a concern that an infectious disease, such as a new coronavirus (covid-19), may cause social economic stagnation. One measure of addressing this concern is to verify that each individual participating in an economic activity is not suffering from the infectious disease.

Also, in a situation where spread of the infectious disease is a concern, there is a great need for non-contact authentication means such as biometric authentication. However, in provision of the service using biometric authentication, a definite mechanism has not been established how to certify non-affection of a user against the infectious disease.

A primary object of the present disclosure is to provide a cooperation server, a system, an immune certificate generation method, and a non-transitory computer-readable medium that contribute to verify that a user is not suffering from an infectious disease.

### Solution to Problem

According to a first aspect of the present disclosure, there is provided a cooperation server including: a reception unit configured to receive a request for generating an immune certificate for an infectious disease; a transmission unit configured to transmit an identity verification request relating to an applicant who has made a request for generating the immune certificate, to an authentication server storing information relating to each of a plurality of users whose identities are confirmed; and a generation unit configured to acquire infectious disease response information relating to an applicant of personal identification information, by transmitting the personal identification information to a server of a public institution, the personal identification information being information uniquely defining the applicant, being identification information issued by the public institution, and being acquired at a time of responding to the identity verification request, and also configured to generate the immune certificate, based on the acquired infectious disease response information.

According to a second aspect of the present disclosure, there is provided a system including: a user terminal configured to transmit a request for generating an immune certificate for an infectious disease; an authentication server configured to store information relating to each of a plurality of users whose identities are confirmed; and a cooperation server being connected to the user terminal and the authentication server, wherein the cooperation server includes: a reception unit configured to receive a request for generating the immune certificate; a transmission unit configured to transmit, to the authentication server, an identity verification request relating to an applicant who has made a request for generating the immune certificate; and a generation unit configured to acquire infectious disease response information relating to an applicant of personal identification information, by transmitting the personal identification information to a server of a public institution, the personal identification information being information uniquely defining the applicant, being identification information issued by the public institution, and being acquired at a time of responding to the identity verification request, and also configured to generate the immune certificate, based on the acquired infectious disease response information.

According to a third aspect of the present disclosure, there is provided an immune certificate generation method including: receiving a request for generating an immune certificate for an infectious disease; transmitting an identity verification request relating to an applicant who has made a request for generating the immune certificate, to an authentication server configured to store information relating to each of a plurality of users whose identities are confirmed; acquiring infectious disease response information relating to an applicant of personal identification information, by transmitting the personal identification information to a server of a public institution, the personal identification information being information uniquely defining the applicant, being identification information issued by the public institution, and being acquired at a time of responding to the identity verification request; and generating the immunity certificate, based on the acquired infectious disease response information.

According to a fourth aspect of the present disclosure, there is provided a non-transitory computer-readable medium that is readable by a computer, and that stores a program for causing a computer mounted on a cooperation server to execute: processing of receiving a request for generating an immune certificate for an infectious disease; processing of transmitting an identity verification request relating to an applicant who has made a request for generating the immune certificate, to an authentication server storing information relating to each of a plurality of users whose identities are confirmed; processing of acquiring infectious disease response information relating to an applicant of personal identification information, by transmitting the personal identification information to a server of a public institution, the personal identification information being information uniquely defining the applicant, being identification information issued by the public institution, and being acquired at a time of responding to the identity verification request; and processing of generating the immune certificate, based on the acquired infectious disease response information.

### Advantageous Effects of Invention

According to each of the aspects of the present disclosure, a cooperation server, a system, an immune certificate generation method, and a non-transitory computer-readable medium are provided that contribute to verify that a user is not suffering from an infectious disease. The advantageous effect of the present disclosure is not limited to the above. Other effects may be achieved in place of the advantageous effect or in conjunction with the advantageous effect according to the present disclosure.

### BRIEF DESCRIPTION OF DRAWINGS

FIG. 1 is a diagram for explaining an outline of an example embodiment;

FIG. 2 is a diagram illustrating an example of a schematic configuration of an authentication system according to a first example embodiment;

FIG. 3 is a diagram illustrating an example of an internal configuration of a processing configuration of a user terminal according to the first example embodiment;

FIG. 4 is a diagram illustrating an example of an internal configuration of a processing configuration of a management server according to the first example embodiment;

FIG. 5 is a diagram illustrating an example of an internal configuration of a processing configuration of an authentication terminal according to the first example embodiment;

FIG. 6 is a diagram illustrating an example of an internal configuration of a processing configuration of an authentication server according to the first example embodiment;

FIG. 7 is a diagram illustrating an example of an authentication information database according to the first example embodiment;

FIG. 8 is a diagram illustrating an example of an internal configuration of a processing configuration of a cooperation server according to the first example embodiment;

FIG. 9 is a sequence diagram illustrating an example of an operation of the authentication system according to the first example embodiment;

FIG. 10 is a diagram illustrating an example of a hardware configuration of a cooperation server; and

FIG. 11 is a diagram illustrating an example of a display of a user terminal.

### EXAMPLE EMBODIMENT

First, an outline of an example embodiment will be described. Note that reference numerals in the drawings attached to this outline are attached to each element for convenience as an example for facilitating understanding, and description of this outline is not intended to be limiting in any way. In addition, unless otherwise specified, blocks described in each drawing do not represent a configuration of a hardware unit, but represent a configuration of a function unit. Connection lines between the blocks in each figure include both bidirectional and unidirectional ones. A one-way arrow schematically illustrates a flow of a main signal (data), and does not exclude bidirectionality. In the present specification and the drawings, the same reference numerals are assigned to elements that can be similarly explained, and a repetitive description thereof can be omitted.

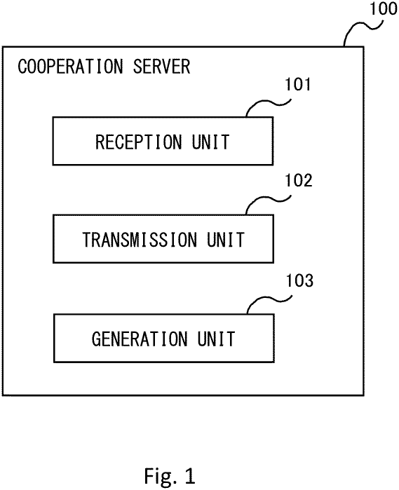

A cooperation server 100 according to an example embodiment includes a reception unit 101, a transmission unit 102, and a generation unit 103 (refer to FIG. 1). The reception unit 101 receives a request for generating an immune certificate for an infectious disease. The transmission unit 102 transmits an identity verification request relating to an applicant who has made a request for generating an immune certificate, to an authentication server that stores information relating to each of a plurality of users whose identities are confirmed. The generation unit 103 transmits, to a server of a public institution, personal identification information, which is information uniquely defining the applicant and is identification information issued by the public institution, the personal identification information being acquired at a time of response to the identity verification request, thereby acquiring infectious disease response information relating to an applicant of the personal identification information. The generation unit 103 generates an immune certificate, based on the acquired infectious disease response information.

The cooperation server 100 achieves cooperation between an authentication server that mainly stores and manages biometric information, personal information, and the like of a user, and a server of a public institution that mainly manages information such as infectious diseases of the user. The cooperation server 100 generates an immune certificate that certifies safety of an infectious disease of a user by utilizing information (personal identification information, e.g., a personal number) held by an authentication server capable of verifying identity of the user and information (infectious disease response information) held by a server of a public institution. The immune certificate verifies (secures) that the user is not suffering from an infectious disease. The user presents the immune certificate to a service provider, such as when entering a place where many people are crowded in a small space. The service provider can deny entrance of an infected person or the like by checking the presented immune certificate. In other words, in a place where only those users who have presented the correct immune certificate gather, a risk of infecting an infectious disease is low. Therefore, the user can participate in social and economic activities with peace of mind.

As for specific example embodiment, explanation will be further made below in detail with reference to the drawings.

### [First Example Embodiment]

A first example embodiment will be explained in more detail by using the drawings.

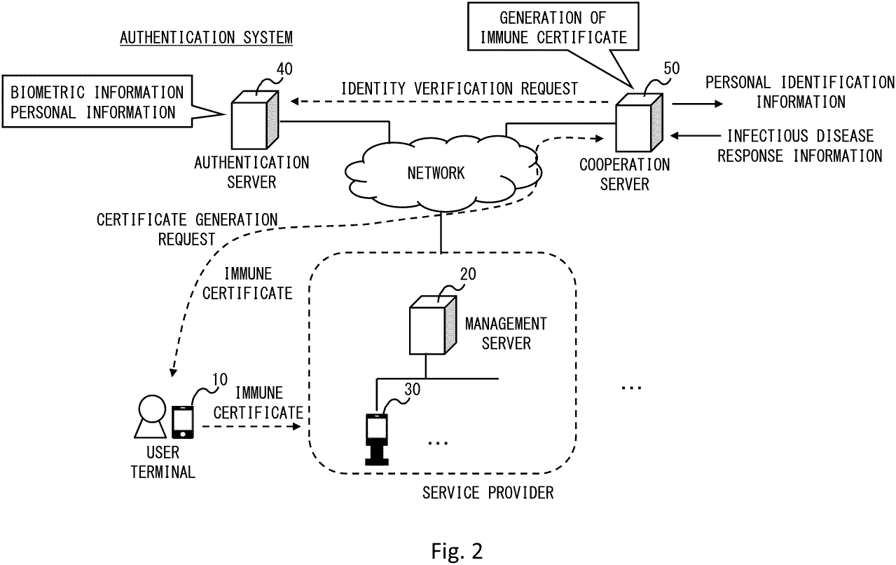

FIG. 2 is a diagram illustrating an example of a schematic configuration of an authentication system according to the first example embodiment. Referring to FIG. 2, the authentication system includes a user terminal 10, a management server 20, an authentication terminal 30, an authentication server 40, and a cooperation server 50.

The apparatuses illustrated in FIG. 2 are connected by a wired or wireless communication means, and are configured to be able to communicate with each other.

The user terminal 10 is a terminal owned by a user of the authentication system. As the user terminal 10, a portable terminal apparatus, such as a smart phone, a cellular phone, a game machine, or a tablet, and the like are exemplified.

The user inputs various types of information into the system via the user terminal 10, or acquires various types of information from the system or the service provider.

The service provider is an operator that provides various services and the like to system users. For example, a hotel operator or a retailer is exemplified as a service provider. The service provider installs and manages the management server 20 and the authentication terminal 30.

Although FIG. 2 illustrates one service provider, a plurality of service providers are actually included in the system.

The management server 20 is a server that controls and manages the entire operations of the service provider. For example, when the service provider is a retail store, the management server 20 performs inventory management of commodities and the like. Alternatively, when the service provider is a hotel operator, the management server 20 manages reservation information of guests.

The authentication terminal 30 is a terminal serving as an interface of a user who has visited a service provider. The user receives various services provided via the authentication terminal 30. For example, when the service provider is a retail store, the user settles the price by using the authentication terminal 30. Alternatively, when the service provider is a hotel operator, the user performs a check-in procedure by using the authentication terminal 30.

The authentication server 40 provides authentication using biometric information. The authentication server 40 operates as a certificate authority in the authentication system.

As biometric information of the user, for example, data (feature amounts) calculated from physical features unique to an individual, such as a face, a fingerprint, a voiceprint, a vein, a retina, and a pattern of an iris of a pupil, are exemplified. Alternatively, the biometric information of the user may be image data of a face image, a fingerprint image, or the like. The biometric information of the user may include the physical characteristics of the user as information.

The authentication server 40 is a server for achieving biometric authentication. The authentication server 40 processes a “authentication request” transmitted from the service provider, and transmits an authentication result to the service provider.

The authentication server 40 stores the biometric information of the user (e.g., a face image or a feature amount generated from the face image) and personal information thereof (e.g., name, date of birth, sex, contact address, personal number, passport number, etc.) in association with each other.

The authentication server 40 also functions as a server that holds information for the user to act in a city. In other words, the authentication server 40 is a server apparatus that stores information for a private sector (service provider) to provide a service to the user.

When registering a user’s system, the authentication server 40 confirms the identity of the user by using an identification document such as a passport or a My Number card of the user. The authentication server 40 stores biometric information and personal information with respect to a user whose identity is confirmed. Namely, the information registered in the authentication server 40 is valid information in which identity confirmation has been conducted. The authentication server 40 stores information relating to each of a plurality of users whose identities are confirmed.

The cooperation server 50 is a server that cooperates private data (data stored in the authentication server 40) with data owned by public institutions (e.g., health centers, Ministry of Health, Labour and Welfare, Ministry of Foreign Affairs, and Ministry of Internal Affairs and Communications) (data managed by ministries and agencies, etc.), and improves user convenience. The cooperation server 50 cooperates with the data (private data) owned by the authentication server 40 and the data (public data) owned by the public institution, and provides various information and services to the user (user terminal 10).

One of the services to be provided by the cooperation server 50 to the user is provision of an “immune certificate”. The immune certificate (negative certificate) is a certificate (electronic certificate) certifying that the user is not suffering from an infectious disease such as a new type of coronavirus (covid-19) infectious disease, for example. The immune certificate includes information relating to a name of a target person (the user who has received issuance of the certificate), a type of infectious disease to certify non-infection, the effective period of the certificate, and the like.

The cooperation server 50 acquires a request for generating an immune certificate (hereinafter, referred to as a certificate generation request) from the user (user terminal 10). When the certificate generation request is acquired, the cooperation server 50 transmits, to the authentication server 40, the “identity verification request” including the name, date of birth, and the like of the user (applicant) who has requested the generation of the immune certificate. In short, the cooperation server 50 transmits information (name of the applicant, etc.) including the applicant who has made the request for generating the immune certificate to the authentication server 40, and requests the authentication server 40 to confirm the identity of the applicant (identity verification).

The authentication server 40 processes the acquired identity verification request. More specifically, the authentication server 40 determines that the identity verification has been successful when the information of the user specified from the name, date of birth, and the like included in the identity verification request is registered in the database. Namely, the information registered in the authentication server 40 is information for which identification (identity confirmation) is completed by the identification document, and the fact that the information is registered in the authentication server 40 means that validity of the information is secured.

When the authentication server 40 succeeds in the identity verification, the authentication server 40 transmits, to the cooperation server 50, identification information which is information uniquely defining the associated user and which is issued by the public institution. The authentication server 40 adds the identification information (e.g., personal number) to an acknowledgement of the identity verification request, and transmits the identification information to the cooperation server 50. In the following explanation, identification information, which is information uniquely defining the user and which is issued by a public institution, is referred to as “personal identification information”.

The cooperation server 50 acquires information for generating an immune certificate from an external server, in particular, a server operated and managed by a public institution. The cooperation server 50 transmits an “infectious disease response information provision request” to the server of the public institution. More specifically, the cooperation server 50 transmits an infectious disease response information provision request including the personal number of the certificate generation target person (applicant) to the external server.

The cooperation server 50 acquires “infectious disease response information” as a response to the request. For example, the cooperation server 50 acquires the infectious disease test information relating to the applicant from the public institution. More specifically, the cooperation server 50 acquires a test result regarding whether or not the applicant is suffering from a new type of coronavirus infectious disease or the like from a public institution (a server installed in the public institution). The cooperation server 50 acquires details on a Polymerase Chain Reaction (PCR) test, an antibody test, and an antigen test for an applicant from the server of the public institution. For example, the cooperation server 50 acquires information such as a type of target infectious disease, a date and time when the applicant takes a PCR test, a location, a result (positive or negative), a PCR test history, and the like.

Alternatively, the cooperation server 50 may acquire an applicant’s status of inoculation (vaccination) for infectious disease from a public institution as “infectious disease response information”. For example, the cooperation server 50 acquires information such as a type of the target infectious disease, a name of the vaccine that has been vaccinated, and a date of vaccination from the public institution.

The cooperation server 50 generates an immune certificate, based on the infectious disease response information (infectious disease test information and infectious disease prevention information) acquired from the public institution. The cooperation server 50 provides the generated immune certificate to the user terminal 10.

The user utilizes the acquired immune certificate in various scenes. For example, a user may present an immune certificate to a hotel operator (service provider) when reserving a hotel and use it to verify that he or she is not suffering from an infectious disease. In addition, the user presents the immune certificate to the hotel operator when checking in the hotel. The hotel operator completes the check-in procedure after confirming that a guest is not suffering from the infectious disease by the presented immune certificate. In other words, when the guest is unable to present a valid immune certificate, the hotel operator may refuse to provide service to the guest.

Alternatively, when the service provider is a business provider that provides an event in a concert hall or the like, the service provider may allow a user who has presented the valid immune certificate to pass through a gate, and may reject a user who is unable to present the valid immune certificate to pass through the gate.

Note that the configuration illustrated in FIG. 2 is an example and is not intended to limit the configuration of the system. For example, all or part of the functions of the cooperation server 50 may be implemented in the authentication server 40.

Next, each apparatus included in the authentication system according to the first example embodiment will be described in detail.

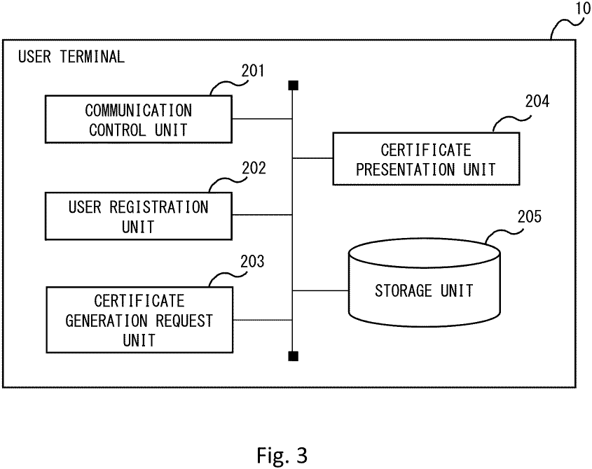

FIG. 3 is a diagram illustrating an example of the internal configuration of a processing configuration (processing module) of the user terminal 10. Referring to FIG. 3, a communication control unit 201, a user registration unit 202, a certificate generation request unit 203, a certificate presentation unit 204, and a storage unit 205 are provided.

The communication control unit 201 is a means for controlling communication with other devices. For example, the communication control unit 201 receives data (packets) from the cooperation server 50. In addition, the communication control unit 201 transmits data to the cooperation server 50. The communication control unit 201 transfers data received from another apparatus to another processing module. The communication control unit 201 transmits data acquired from another processing module to another apparatus. As described above, another processing module transmits and receives data to and from another device via the communication control unit 201.

The user registration unit 202 is a means for performing user registration relating to the use of the authentication system. The user registration unit 202 acquires biometric information (e.g., face image) of the user, personal information (e.g., name, date of birth, sex, contact address, etc.) thereof, a copy of an identification document (e.g., My Number card, passport), and the like by using a Graphical User Interface (GUI) or the like.

The user registration unit 202 transmits the acquired information (biometric information, personal information, and identification document) to the authentication server 40.

The certificate generation request unit 203 is a means for requesting the cooperation server 50 to generate an immune certificate. The certificate generation request unit 203 transmits a “certificate generation request” including information (e.g., name, date of birth, etc.) for specifying the applicant (owner of the user terminal 10) to the cooperation server 50.

When the certificate generation request unit 203 acquires the immune certificate from the cooperation server 50, the certificate generation request unit 203 stores the acquired immune certificate in the storage unit 205.

The certificate presentation unit 204 is a means for presenting an immune certificate to a third party. The certificate presentation unit 204 presents the immune certificate spontaneously or in response to a request from a third party.

For example, a case where the service provider is a hotel operator is considered. In this case, the user accesses a Web site operated and managed by the hotel operator by using the user terminal 10. For example, the management server 20 operates as the Web server.

The certificate presentation unit 204 reads out the immune certificate stored in the storage unit 205 and provides (presents) the immune certificate to the management server 20 when the management server 20 requests presentation of the immune certificate at a time of accommodation reservation. The management server 20 permits the user’s reservation procedure when a valid immune certificate is presented.

The certificate presentation unit 204 may present the immune certificate to a third party by a short-range wireless communication means such as Bluetooth (registered trademark). For example, the case where the service provider is a hotel operator is considered as in the above example. When the reservation is already completed and the day of stay comes, the user visits the hotel where the reservation has been made. When the user performs the check-in procedure by using the authentication terminal 30, the certificate presentation unit 204 may present the immune certificate to the authentication terminal 30. For example, when a distance between the user terminal 10 and the authentication terminal 30 becomes equal to or less than a predetermined distance, the certificate presentation unit 204 may present the immune certificate to the authentication terminal 30. The authentication terminal 30 permits the user’s operation (check-in procedure using the authentication terminal 30) when a valid immune certificate is presented.

The certificate presentation unit 204 may present the immune certificate by using a camera or the like installed by the service provider. For example, a case where a gate apparatus is installed at an entrance of a service provider or the like is considered. When the gate apparatus and a camera apparatus are connected to each other, the certificate presentation unit 204 generates a two-dimensional bar code including the content of the immune certificate, and displays the two-dimensional barcode on a display unit (liquid crystal panel, etc.). The user brings the displayed two-dimensional bar code (immune certificate) closer to the camera apparatus. The gate apparatus acquires the content of the immune certificate from the camera apparatus, and allows the user to pass (enter) when the immune certificate is valid.

The storage unit 205 is a means for storing information necessary for an operation of the user terminal 10.

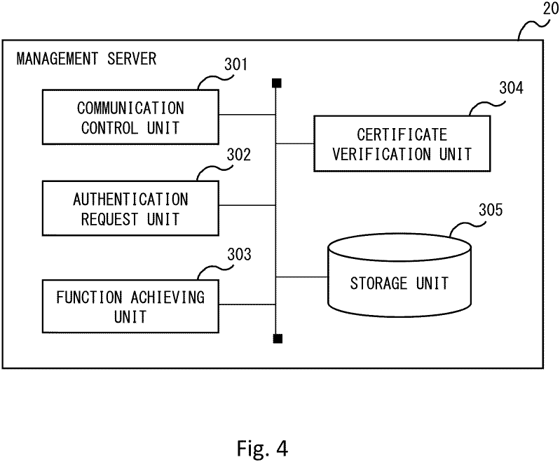

FIG. 4 is a diagram illustrating an example of a processing configuration (processing module) of the management server 20. Referring to FIG. 4, the management server 20 includes a communication control unit 301, an authentication request unit 302, a function achieving unit 303, a certificate verification unit 304, and a storage unit 305.

The communication control unit 301 is a means for controlling communication with another device. For example, the communication control unit 301 receives data (packets) from the authentication server 40. The communication control unit 301 transmits data to the authentication server 40. The communication control unit 301 transfers data received from another apparatus to another processing module. The communication control unit 301 transmits data acquired from another processing module to another apparatus. As described above, the another processing module transmits and receives data to and from the another apparatus via the communication control unit 301.

The authentication request unit 302 is a means for requesting biometric authentication of the user for the authentication server 40. When the authentication request unit 302 acquires biometric information (face image) of the user from the authentication terminal 30, the authentication request unit 302 generates a feature amount from the face image. The authentication request unit 302 transmits an authentication request including the generated feature amount (biometric information), to the authentication server 40.

The authentication request unit 302 receives the authentication result from the authentication server 40. When the authentication request unit 302 acquires an authentication success (acknowledgement), the authentication request unit 302 transfers a name of an authenticated person included in the response to the function achieving unit 303. When the authentication request unit 302 acquires an authentication failure (negative acknowledgement) from the authentication server 40, the authentication request unit 302 notifies the authenticated person of the authentication failure.

The function achieving unit 303 is a means for achieving a function of each service provider. When the service provider is a hotel operator, the function achieving unit 303 performs reservation processing of the user and a check-in procedure thereof. For example, when the reservation processing of the user is performed, the function achieving unit 303 provides a GUI or an input form for acquiring reservation information such as a name, an address, and an accommodation date via the user terminal 10. The function achieving unit 303 stores the information acquired from the user in the storage unit 305 as reservation information.

When the check-in procedure of the user is performed, the function achieving unit 303 searches reservation information by using a name of a guest acquired via the authentication request unit 302, and specifies the reservation information of the guest. The function achieving unit 303 performs a check-in procedure or the like by using the specified reservation information.

When the function achieving unit 303 controls opening and closing of the gate, the function achieving unit 303 opens the gate when the authentication result from the authentication server 40 is “authentication success”.

The function achieving unit 303 acquires an immune certificate from the user (user terminal 10) as necessary. For example, the function achieving unit 303 may request presentation of an immune certificate when information is input on a Web page. Alternatively, the function achieving unit 303 may acquire an immune certificate by short-range wireless communication. Alternatively, the function achieving unit 303 may acquire an immune certificate via a camera apparatus.

The function achieving unit 303 delivers the acquired immune certificate to the certificate verification unit 304.

The certificate verification unit 304 is a means for verifying the validity (effectiveness) of the immune certificate acquired from the user (user terminal 10).

For example, the certificate verification unit 304 verifies whether or not a name described in the immune certificate (the name of the user who is verified as being unaffected by the infectious disease) matches the name of the user who has presented the immune certificate. The certificate verification unit 304 acquires the name and the like of the user who has presented the immune certificate by using various means. For example, the certificate verification unit 304 may use the name being input at a time of reservation of accommodation. Alternatively, the certificate verification unit 304 may transmit the biometric information acquired via the authentication terminal 30 to the authentication server 40, and use the name of the user which is associated to the biometric information. The certificate verification unit 304 may determine that the immune certificate is valid when the two names match.

The certificate verification unit 304 may verify the validity of the immune certificate, based on an effective period described in the immune certificate. The certificate verification unit 304 may determine that the immune certificate is effective when the effective period has not elapsed.

The certificate verification unit 304 may determine that the immune certificate is effective when both results of the verification on the subject of the immune certificate (verification on consistency between a user who certifies non-affection in the immune certificate and a presenter of the immune certificate) and the verification on the effective period of the immune certificate are effective.

The certificate verification unit 304 notifies the function achieving unit 303 of the determination result.

The function achieving unit 303 performs processing, based on the determination result. When the determination result is negative, the function achieving unit 303 rejects the service provision to the user (rejection of reservation, rejection of accommodation, and rejection of gate opening). When the determination result is positive, the function achieving unit 303 provides a service to the user. Thus, when the immune certificate is not effective (e.g., an effective period is expired), service provision by the service provider may not be performed.

The storage unit 305 is a means for storing information necessary for an operation of the management server 20.

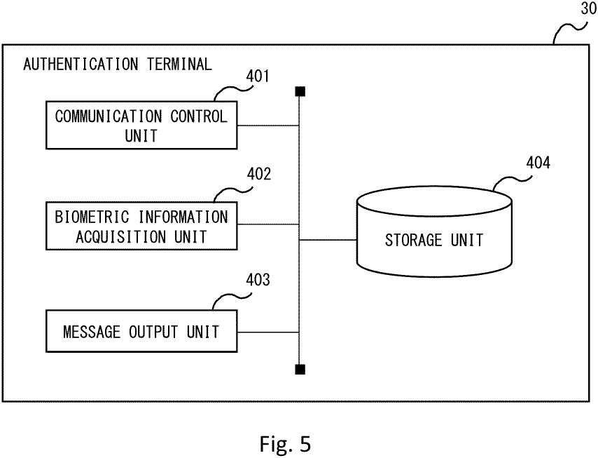

FIG. 5 is a diagram illustrating an example of a processing configuration (processing module) of the authentication terminal 30. Referring to FIG. 5, the authentication terminal 30 includes a communication control unit 401, a biometric information acquisition unit 402, a message output unit 403, and a storage unit 404.

The communication control unit 401 is a means for controlling communication with another apparatus. For example, the communication control unit 401 receives data (packets) from the management server 20. The communication control unit 401 transmits data to the management server 20. The communication control unit 401 transfers data received from another apparatus to another processing module. The communication control unit 401 transmits data acquired from another processing module to another apparatus. As described above, the another processing module transmits and receives data to and from the another apparatus via the communication control unit 401.

The biometric information acquisition unit 402 is a means for acquiring biometric information (e.g., a face image) of a user. The biometric information acquisition unit 402 transmits the acquired biometric information to the management server 20.

The message output unit 403 is a means for outputting various messages by using a device such as a liquid crystal panel or a speaker. The message output unit 403 notifies the user of a message from the management server 20 (a message in response to the authentication result) or a message in the procedure (a message at the time of the check-in procedure).

The storage unit 404 is a means for storing information necessary for an operation of the authentication terminal 30.

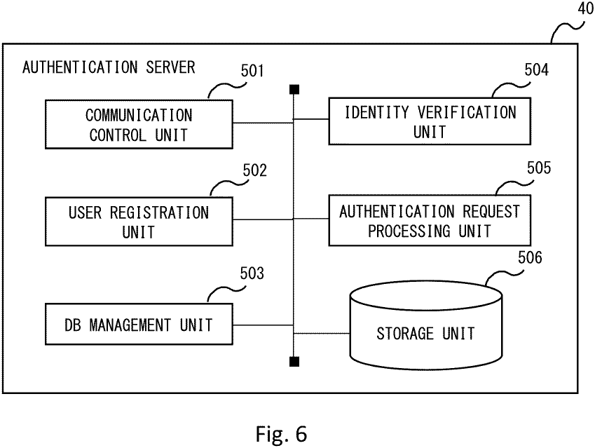

FIG. 6 is a diagram illustrating an example of a processing configuration (processing module) of the authentication server 40. Referring to FIG. 6, the authentication server 40 includes a communication control unit 501, a user registration unit 502, a database management unit 503, an identity verification unit 504, an authentication request processing unit 505, and a storage unit 506.

The communication control unit 501 is a means for controlling communication with other devices. For example, the communication control unit 501 receives data (packets) from the management server 20. The communication control unit 501 transmits data to the management server 20. The communication control unit 501 transfers data received from another apparatus to another processing module. The communication control unit 501 transmits data acquired from another processing module to another apparatus. As described above, the another processing module transmits and receives data to and from the another apparatus via the communication control unit 501.

The user registration unit 502 is a means for achieving system registration of a user. The user registration unit 502 acquires biometric information, personal information, identification documents, and the like from the user (user terminal 10).

When the user registration unit 502 acquires the information, the user registration unit 502 makes an identification (identity confirmation) of the applicant related to the system registration. Specifically, the user registration unit 502 verifies whether or not the acquired biometric information (face image) substantially matches the face image described in the identification document. When the two face images substantially coincide with each other, the user registration unit 502 determines that the identification of the applicant has been successful.

The user registration unit 502 generates feature amounts (feature vectors) from the two face images, and determines that the two face images substantially coincide with each other when a distance between the feature vectors (Euclidean distance, etc.) is larger than a threshold value.

Upon successful identification of the applicant, the user registration unit 502 generates a user ID for uniquely identifying the user. For example, the user registration unit 502 may assign a unique value to the user ID every time the user is registered.

The user registration unit 502 delivers the user ID, biometric information (feature amount, face image), personal information, identification documents, and the like to the database management unit 503.

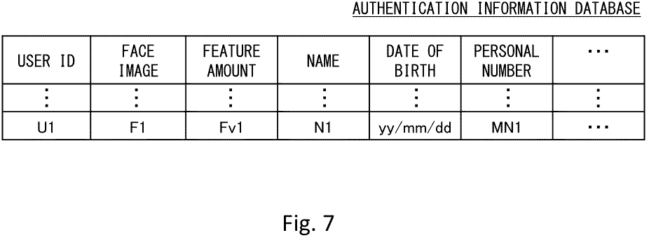

The database (DB: Data Base) management unit 503 is a means for managing an authentication information database. The authentication information database stores user IDs, biometric information, personal information, identification documents, and the like in association with each other (refer to FIG. 7). As illustrated in FIG. 7, the authentication information database stores a user ID, biometric information, personal information (name, personal number, etc.) and the like of the user in association with each other.

It is needless to say that the authentication information database illustrated in FIG. 7 is only an example, and is not intended to limit the stored content or the like. For example, the face image may not be stored in the authentication information database, or other information may be stored therein.

When the database management unit 503 acquires the user ID and the like from the user registration unit 502, the database management unit 503 adds a new entry to the authentication information database and stores the above information.

The identity verification unit 504 is a means for processing an “identity verification request” received from the cooperation server 50. The identity verification request requires verification of whether or not the applicant for the immune certificate is a certain person who has already been identified. The identity verification request includes information specifying a requester, such as a name and date of birth of the issue requester of the immunity certificate.

The identity verification unit 504 searches the authentication information database by using the name, date of birth, and the like extracted from the identity verification request as keys, and specifies an entry. When an associated entry can be found, the identity verification unit 504 determines that the identity verification has succeeded. In this case, the identity verification unit 504 transmits an acknowledgement including the personal identification information (e.g., personal number) included in the specified entry to the cooperation server 50.

When the associated entry cannot be found, the identity verification unit 504 determines that the identity verification has failed. In this case, the identity verification unit 504 transmits a negative acknowledgement to the cooperation server 50.

The authentication request processing unit 505 is a means for processing an authentication request to be acquired from the management server 20. The authentication request processing unit 505 acquires biometric information (feature amount) from the authentication request acquired from the management server 20.

The authentication request processing unit 505 sets the acquired feature amount to a collating side and the feature amount stored in the authentication information database to a registering side, respectively, and executes one-to-N collation (N is a positive integer, hereinafter the same). When, as a result of the collation processing, the feature amount substantially coincident with the feature amount on the collating side is registered in the authentication information database, the authentication request processing unit 505 determines that the authentication has succeeded. In this case, the authentication request processing unit 505 transmits an acknowledgement to the management server 20. As a result of the collation processing, when the feature amount substantially coincident with the feature amount on the collating side is not registered in the authentication information database, the authentication request processing unit 505 determines that the authentication has failed. In this case, the authentication request processing unit 505 transmits a negative acknowledgement to the management server 20.

When the authentication has succeeded, the authentication request processing unit 505 transmits the personal information of the authenticated person (e.g., name, etc.) to the management server 20 as necessary.

The storage unit 506 is a means for storing information necessary for an operation of the authentication server 40.

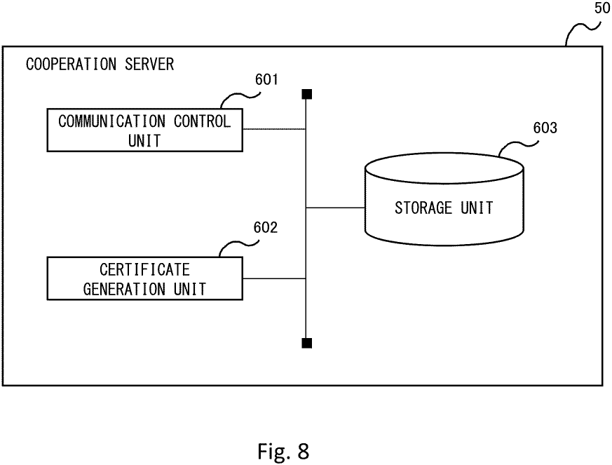

FIG. 8 is a diagram illustrating an example of a processing configuration (processing module) of the cooperation server 50. Referring to FIG. 8, the cooperation server 50 includes a communication control unit 601, a certificate generation unit 602, and a storage unit 603.

The communication control unit 601 is a means for controlling communication with another apparatus. For example, the communication control unit 601 receives data (packets) from the authentication server 40. The communication control unit 601 transmits data to the authentication server 40. The communication control unit 601 transfers data received from another apparatus to another processing module. The communication control unit 601 transmits data acquired from another processing module to another apparatus. As described above, the another processing module transmits and receives data to and from the another apparatus via the communication control unit 601. The communication control unit 601 has a function as a reception unit that receives a certificate generation request and a function as a transmission unit that transmits an identity verification request.

The certificate generation unit 602 is a means for processing a “certificate generation request” from the user terminal 10. The certificate generation unit 602 acquires information that can specify the applicant, such as the name and date of birth of the applicant, from the certificate generation request acquired from the user terminal 10.

The certificate generation unit 602 generates an “identity verification request” including information such as the acquired name and date of birth, and transmits the identity certification request to the authentication server 40.

The certificate generation unit 602 transmits, to a public institution, an “infectious disease response information provision request” including information (personal identification information; e.g., a personal number) specifying the user (applicant) whose identity has been verified by the authentication server 40. More specifically, the certificate generation unit 602 transmits the above request to a server installed in the public institution.

The certificate generation unit 602 acquires a response to the infectious disease response information provision request from the server of the public institution. The response includes detailed information on the applicant’s test for infectious disease (referred to as infectious disease test information).

The certificate generation unit 602 generates an immune certificate, based on the acquired infectious disease test information. For example, the certificate generation unit 602 issues an immune certificate to an applicant for whom a PCR test and an antibody test for an infectious disease have been performed and a negative result has been confirmed. At this time, the certificate generation unit 602 may set an effective period in response to a period from the confirmation of the negative result to an application date. For example, when a long period of time has elapsed since the confirmation of negative result, the certificate generation unit 602 sets the effective period to be short.

The certificate generation unit 602 may generate an immune certificate, based on information regarding vaccination against infectious diseases (infectious disease prevention information). The certificate generation unit 602 transmits, for example, an infectious disease response information provision request to a public institution, and acquires infectious disease prevention information as a response thereto. For example, the certificate generation unit 602 issues an immune certificate to an applicant who has been vaccinated with an effective vaccine against an infectious disease. Also in this case, the certificate generation unit 602 may set an effective period, based on the elapsed time from a vaccination date or the like.

The certificate generation unit 602 generates an immune certificate including the type of the target infectious disease, the name of the user whose immune certificate certifies non-affection of the infectious disease (the name of the issue applicant of the immune certificate), the effective period, and the like, and transmits the generated immune certificate to the user terminal 10. The certificate generation unit 602 transmits the generated immune certificate to the user terminal 10, which is a transmission source of the certificate generation request, via the communication control unit 601.

As described above, the certificate generation unit 602 transmits the personal identification information uniquely defining the applicant (identification information issued by the public institution, e.g., a personal number), which is acquired at the time of response to the identity verification request to the server of the public institution. The certificate generation unit 602 acquires the infectious disease response information relating to the applicant of the personal identification information by transmitting the personal identification information. The certificate generation unit 602 generates an immune certificate, based on the acquired infectious disease response information.

The storage unit 603 is a means for storing information necessary for an operation of the cooperation server 50.

The explanation of an external server of a public institution is omitted. Officials of the public institution enter information on infected persons (infectious disease test information, infectious disease prevention information) and personal numbers of infected persons into the server. In response to a request from the cooperation server 50, the server may search database by using the personal number as a key, and respond associated information.

### [System Operation]

Next, an operation of the authentication system according to the first example embodiment will be described. The operation is explained with respect to issuance of an immune certificate, and explanations of user registration and presentation of the immune certificate are omitted.

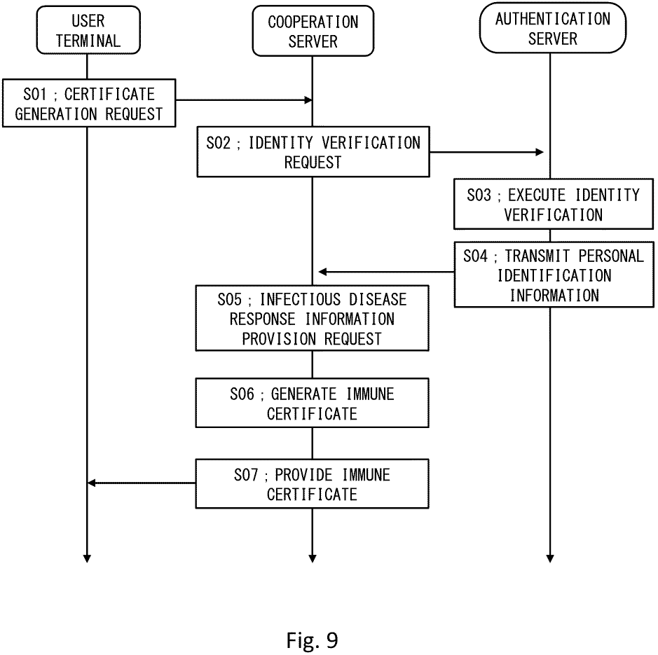

FIG. 9 is a sequence diagram illustrating an example of an operation relating to issuance of an immune certificate of the authentication system according to the first example embodiment.

The user terminal 10 transmits a “certificate generation request” to the cooperation server 50 (step S01).

The cooperation server 50 transmits an identity verification request to the authentication server 40 (step S02).

The authentication server 40 confirms the identity of the applicant for issuance of the immune certificate by searching the authentication information database (execution of identity verification: step S03).

When the authentication server 40 succeeds in the identity verification, the authentication server 40 transmits a response including personal identification information (e.g., a personal number) of the applicant to the cooperation server 50 (step S04).

The cooperation server 50 transmits infectious disease response information provision request including the personal identification information (e.g., a personal number) to an external server (a server of a public institution) (step S05). The cooperation server 50 acquires infectious disease response information (infectious disease test information and infectious disease prevention information) as a response to the request from the external server.

The cooperation server 50 generates an immune certificate, based on the acquired infectious disease response information (step S06).

The cooperation server 50 provides (issues) the generated immune certificate to the user terminal 10 (step S07).

As described above, in the authentication system according to the first example embodiment, the immune certificate is issued in response to a request from the user. The user (user terminal 10) presents the immune certificate to the service provider, thereby certifying non-affection of the infectious disease. The service provider can provide a service to a user who is not suffering from an infectious disease and deny the service provision to the user who is suffering from the infectious disease. As a result, the user who is not suffering from the infectious disease can participate in social and economic activities with peace of mind.

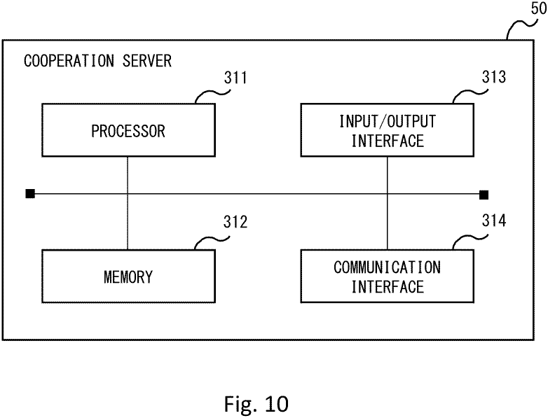

Next, hardware of each apparatus constituting the authentication system will be explained. FIG. 10 is a diagram illustrating an example of a hardware configuration of the cooperation server 50.

The cooperation server 50 can be configured by an information processing apparatus (so-called a computer), and has a configuration exemplified in FIG. 10. For example, the cooperation server 50 includes a processor 311, a memory 312, an input/output interface 313, a communication interface 314, and the like. The components such as the processor 311 are connected by an internal bus or the like, and are configured to be able to communicate with each other.

However, the configuration illustrated in FIG. 10 is not intended to limit the hardware configuration of the cooperation server 50. The cooperation server 50 may include unillustrated hardware, or may not include the input/output interface 313 as necessary. The number of processors 311 and the like included in the cooperation server 50 is not intended to be limited to the example of FIG. 10, and for example, a plurality of processors 311 may be included in the cooperation server 50.

The processor 311 is, for example, a programmable device such as a Central Processing Unit (CPU), a Micro Processing Unit (MPU), or a Digital Signal Processor (DSP). Alternatively, the processor 311 may be a device such as a Field Programmable Gate Array (FPGA) or an Application Specific Integrated Circuit (ASIC). The processor 311 executes various programs including an operating system (OS).

The memory 312 is a Random Access Memory (RAM), Read Only Memory (ROM), Hard Disk Drive (HDD), Solid State Drive (SSD), or the like. The memory 312 stores an OS program, an application program, and various data.

The input/output interface 313 is an interface of a display apparatus or an input apparatus which is not illustrated. The display apparatus is, for example, a liquid crystal display or the like. The input apparatus is, for example, an apparatus that accepts a user operation such as a keyboard or a mouse.

The communication interface 314 is a circuit, a module, or the like that performs communication with another apparatus. For example, the communication interface 314 includes a network interface card (NIC) or the like.

A function of the cooperation server 50 is achieved by various processing modules. The processing module is achieved, for example, by the processor 311 executing a program stored in the memory 312. The program can be recorded on a computer-readable medium that is readable by a computer. The computer-readable medium may be a non-transitory medium, such as a semiconductor memory, a hard disk, a magnetic recording medium or an optical recording medium. Namely, the present disclosure may be embodied as a computer program product. The program may be downloaded via a network or updated by using a computer-readable medium having the program stored thereon. Further, the above processing module may be achieved by a semiconductor chip.

Note that the user terminal 10, the management server 20, the authentication terminal 30, the authentication server 40, and the like can also be configured by an information processing apparatus similarly to the cooperation server 50, and the basic hardware configuration thereof is not different from that of the cooperation server 50, and therefore description thereof is omitted.

The function of the cooperation server 50 can be achieved by mounting a computer thereon and causing the computer to execute a program. In addition, the cooperation server 50 executes an immune certificate generation method by the program.

### [Modified Example]

Note that the configuration, operation, and the like of the authentication system which are explained in the above example embodiment are exemplified, and are not intended to limit the configuration and the like of the system.

The certificate generation unit 602 of the cooperation server 50 may attach an electronic signature to the generated immune certificate and provide the resultant immune certificate to the user terminal 10. The certificate verification unit 304 of the management server 20 may verify the electronic signature attached to the immune certificate. The certificate verification unit 304 may accept the presented immune certificate when the verification of the electronic signature is successful. Namely, the certificate verification unit 304 may verify the validity of the issuer of the immune certificate and the fact that the content of the immune certificate has not been tampered with.

In the above example embodiment, the case where biometric information related to the “feature amount generated from the face image” is transmitted from the management server 20 to the authentication server 40 has been described. However, the biometric information related to the “face image” may be transmitted from the management server 20 to the authentication server 40. In this case, the authentication server 40 may generate the feature amount from the acquired face image and execute the authentication processing (collation processing).

In the above example embodiment, cancellation of the immune certificate issued once is not mentioned, but the cooperation server 50 may cancel the immune certificate when a predetermined condition is satisfied. For example, when it is found that a user who has received issuance of an immune certificate is a close contact person of an infected person, an immune certificate cancellation notification may be transmitted to the user terminal 10. The user terminal 10, which has received the notification, deletes the immune certificate stored in the storage unit 205. Alternatively, the cooperation server 50 may shorten the effective period of the immune certificate issued to the user when the user is found to be a close contact person.

In the above example embodiment, the case where the user acquires the immune certificate in advance when the presentation of the immune certificate is expected has been explained. However, the user (user terminal 10) may acquire an immune certificate in response to a request from a service provider or the like. For example, a case where a user makes a reservation for accommodation is considered. In this case, the user performs a reservation procedure as usual on the Web page of the hotel operator, or the like. When the hotel operator determines that the presentation of the immune certificate is necessary in response to a situation such as an epidemic period of the infectious disease, the hotel operator notifies the user terminal 10 of that effect. In response to the notification, the user terminal 10 may acquire an immune certificate from the cooperation server 50 and present the acquired immune certificate to the hotel operator (management server 20). The hotel operator may permit the reservation when a negative result of the reservation person is confirmed by the immune certificate. The hotel operator may make a similar request during the check-in procedure.

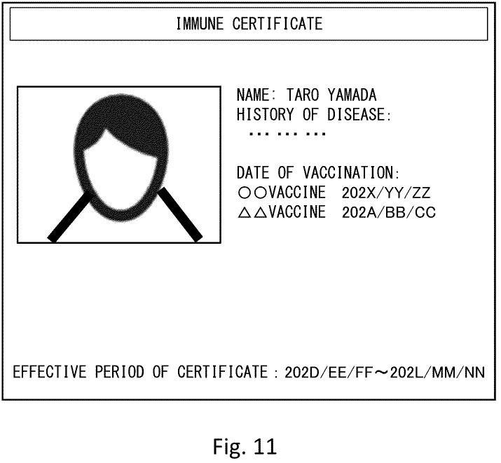

In the above example embodiment, the case where the user terminal 10 presents the electronic immune certificate to the apparatus and the device has been described. However, the user terminal 10 can also present an immune certificate to a person. For example, the user terminal 10 may display the immune certificate on a liquid crystal panel or the like when the service provider’s store clerk, employee, or the like requests the presentation of the immune certificate. In this case, the user operates the user terminal 10 and presents a display as illustrated in FIG. 11 to a store clerk or the like. The store clerk or the like verifies the display of FIG. 11 and verifies that the presenter of the immune certificate is not suffering from an infectious disease.

Alternatively, the service provider may inquire of the cooperation server 50 or the like about the validity of the immune certificate presented by the display. The user terminal 10 displays a two-dimensional bar code generated (converted) from information (e.g., a personal number) that can confirm the identity of the user, together with an immune certificate. The capacity of the service provider reads the two-dimensional bar code by using the terminal, and transmits the content to the cooperation server 50. When it is possible to verify that the user is not suffering from an infectious disease by using his/her personal number, the cooperation server 50 returns that effect to the service provider. When an acknowledgement is returned, the service provider permits the entry or the like of the user. Such measures can prevent unauthorized use or the like of a forged immune certificate.

As explained in the above example embodiment, when the service provider is a hotel operator, the service provider (management server 20) may request the user to present the immune certificate at the time of reservation and at the time of accommodation. Namely, the user terminal 10 acquires the immune certificate from the cooperation server 50 at the time of reservation of accommodation, and presents the acquired immune certificate to the hotel business operator. The hotel operator permits the reservation when a valid immune certificate could have been acquired from the user. In addition, when the user is using the hotel, the immune certificate is acquired from the cooperation server 50 before the user visits the hotel (the immune certificate is downloaded to the user terminal 10). The user terminal 10 temporarily stores the immune certificate (effective immune certificate). When the user visits the hotel, the user terminal 10 presents the temporarily stored immune certificate to a check-in terminal (authentication terminal 30). The hotel operator permits the check-in when the validity of the immune certificate presented at the time of the check-in can be confirmed.

In the above example embodiment, the case where the immune certificate issued by the cooperation server 50 is stored (downloaded) in the user terminal 10 has been described, but the immune certificate may be stored in another apparatus. For example, the authentication server 40 may store an immune certificate in association with biometric information, personal information, or the like. When biometric authentication of the user is successful, the authentication server 40 may transmit the immune certificate together with the authentication result (authentication success) to the management server 20 or the like. With such a response, it is possible to automatically verify presence or absence of infection to be performed on a successfully authenticated person. For example, it is assumed that a gate apparatus transmits an authentication request to the authentication server 40. In this case, the authentication server 40 transmits the immune certificate of the successfully authenticated person to the gate apparatus together with the authentication result (authentication success). The gate apparatus may open the gate on condition that authentication of an authenticated person is successful and that the immune certificate of the successfully authenticated person is effective. Alternatively, instead of the authentication server 40, another intermediate server may store the immune certificate.

In the above example embodiment, it has been explained that the name and the like of the user who certifies non-affection of the infectious disease are described in the immune certificate. The immune certificate may include other information. For example, the immune certificate may include biometric information (face image or feature amount) of a certified person. In this case, the verification of the immune certificate may be performed depending on whether or not the biometric information described in the immune certificate substantially matches the biometric information of a presenter of the immune certificate. Namely, the certificate verification unit 304 of the management server 20 may verify the immune certificate, based on a result of one-to-one collation using two face images as targets. The certificate verification unit 304 may determine that the immune certificate is valid (effective) when the two face images match as a result of the collation.

Alternatively, the immune certificate may include information indicating that the user who has received the issuance of the immune certificate is equivalent to the close contact person, or information on a level of the close contact person (secondary close contact person, tertiary close contact person).

In the above example embodiment, the identity confirmation (authentication) to be performed when the immune certificate is utilized is based on biometric authentication, but the identity confirmation may be performed by other methods. For example, it may be identity confirmation using an Identifier (ID) and a password.

The mode of data transmission and reception among the respective apparatuses (user terminal 10, management server 20, authentication terminal 30, authentication server 40, cooperation server 50) is not particularly limited, but data transmitted and received among these apparatuses may be encrypted. It is preferable that biometric information is transmitted and received between these apparatuses, and encrypted data are transmitted and received in order to appropriately protect the biometric information.

In the above example embodiment, the use of an immune certificate has been explained by taking a hotel operator and an event operator as examples. However, the present disclosure is not limited to these industry types, and the use of the immune certificate can be performed in a wide range of industry types and industries. Namely, service providers other than the hotel operator can utilize the immune certificates by checking in, checkout, entry and exit via a gate, and the like.

In the flowcharts (flowcharts, sequence diagrams) used in the above explanation, a plurality of steps (processes) are described in order, but the order of execution of the steps being executed in the example embodiment is not limited to the order of description. In the example embodiment, the order of the illustrated steps can be changed to the extent that there is no problem in the contents, for example, the processes are executed in parallel, and the like.

The above example embodiments have been described in detail in order to facilitate understanding of the present disclosure, and are not intended to require all of the configurations explained above. When a plurality of example embodiments are explained, each example embodiment may be used alone or in combination. For example, a part of the configuration of the example embodiment can be replaced with the configuration of another example embodiment, or the configuration of another example embodiment can be added to the configuration of the example embodiment. In addition, some of the configurations of the example embodiments may be added, deleted, or replaced with other configurations.

Although the industrial applicability of the present disclosure is obvious from the above explanation, the present disclosure can be suitably applied to an authentication system or the like of confirming that a user is not suffering from an infectious disease.

Some or all of the above example embodiments may also be described as the following supplementary notes, but are not limited to the following.

### Supplementary Note 1

A cooperation server comprising:
a reception unit configured to receive a request for generating an immune certificate for an infectious disease;
a transmission unit configured to transmit an identity certification request relating to an applicant who has made a request for generating the immune certificate, to an authentication server storing information relating to each of a plurality of users whose identities are confirmed; and
a generation unit configured to acquire infectious disease response information relating to an applicant of personal identification information by transmitting the personal identification information to a server of a public institution, the personal identification information being information uniquely defining the applicant, being identification information issued by a public institution, and being acquired at a time of responding to the identity verification request, and also configured to generate the immune certificate, based on the acquired infectious disease response information.

### Supplementary Note 2

The cooperation server according to Supplementary note 1, wherein the transmission unit transmits the generated immune certificate to a user terminal being a transmission source of a request for generating the immune certificate.

### Supplementary Note 3

The cooperation server according to Supplementary note 1 or 2, wherein the generation unit generates the immune certificate, based on information on testing of the applicant for an infectious disease or information relating to vaccination of the applicant for an infectious disease.

### Supplementary Note 4

The cooperation server according to any one of Supplementary notes 1 to 3, wherein the generation unit generates the immune certificate including an effective period.

### Supplementary Note 5

The cooperation server according to any one of Supplementary notes 1 to 4, wherein the personal identification information is a personal number.

### Supplementary Note 6

A system comprising:
a user terminal configured to transmit a request for generating an immune certificate for an infectious disease;
an authentication server configured to store information relating to each of a plurality of users whose identities are confirmed; and
a cooperation server being connected to the user terminal and the authentication server, wherein the cooperation server includes
   a reception unit configured to receive a request for generating the immune certificate,
   a transmission unit configured to transmit, to the authentication server, an identity verification request relating to an applicant who has made a request for generating the immune certificate; and
   a generation unit configured to acquire infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request, and also configured to generate the immune certificate, based on the acquired infectious disease response information.

### Supplementary Note 7

The system according to Supplementary note 6, wherein the transmission unit transmits the generated immune certificate to the user terminal being a transmission source of a request for generating the immune certificate.

### Supplementary Note 8

An immune certificate generation method comprising:
receiving a request for generating an immune certificate for an infectious disease;
transmitting an identity verification request relating to an applicant who has made a request for generating the immune certificate, to an authentication server storing information relating to each of a plurality of users whose identities are confirmed;
acquiring infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request; and
generating the immune certificate, based on the acquired infectious disease response information.

### Supplementary Note 9

A non-transitory computer-readable medium that is readable by a computer and stores a program for causing a computer mounted on a cooperation server to execute:
processing of receiving a request for generating an immune certificate for an infectious disease;
processing of transmitting an identity verification request relating to an applicant who has made a request for generating the immune certificate, to an authentication server storing information relating to each of a plurality of users whose identities are confirmed;
processing of acquiring infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request; and
processing of generating the immune certificate, based on the acquired infectious disease response information.

The disclosures of the above cited prior art literatures are incorporated herein by reference. While the example embodiments of the present disclosure have been explained above, the present disclosure is not limited to these example embodiments. It will be appreciated by those skilled in the art that these example embodiments are illustrative only and that various modifications are possible without departing from the scope and spirit of the present disclosure. Namely, the present disclosure includes all the disclosures including the claims and various deformations and modifications that can be made by a person skilled in the art in accordance with the technical idea.

Reference Signs List

| | |
|---|---|
| 10 | USER TERMINAL |
| 20 | MANAGEMENT SERVER |
| 30 | AUTHENTICATION TERMINAL |
| 40 | AUTHENTICATION SERVER |
| 50,100 | COOPERATION SERVER |
| 101 | RECEPTION UNIT |
| 102 | TRANSMISSION UNIT |
| 103 | GENERATION UNIT |
| 201, 301, 401, 501, 601 | COMMUNICATION CONTROL UNIT |
| 202,502 | USER REGISTRATION UNIT |
| 203 | CERTIFICATE GENERATION REQUEST UNIT |
| 204 | CERTIFICATE PRESENTATION UNIT |
| 205, 305, 404, 506, 603 | STORAGE UNIT |
| 302 | AUTHENTICATION REQUEST UNIT |
| 303 | FUNCTION ACHIEVING UNIT |
| 304 | CERTIFICATE VERIFICATION UNIT |
| 311 | PROCESSOR |
| 312 | MEMORY |
| 313 | INPUT/OUTPUT INTERFACE |
| 314 | COMMUNICATION INTERFACE |
| 402 | BIOMETRIC INFORMATION ACQUISITION UNIT |
| 403 | MESSAGE OUTPUT UNIT |
| 503 | DATABASE MANAGEMENT UNIT |
| 504 | IDENTITY VERIFICATION UNIT |
| 505 | AUTHENTICATION REQUEST PROCESSING UNIT |
| 602 | CERTIFICATE GENERATION UNIT |

What is claimed is:

1. A cooperation server comprising:
at least one memory configured to store instructions; and
at least one processor configured to execute the instructions to:
receive a request for generating an immune certificate for an infectious disease;
transmit an identity certification request relating to an applicant who has made a request for generating the immune certificate, to an authentication server storing information relating to each of a plurality of users whose identities are confirmed; and
acquire infectious disease response information relating to an applicant of personal identification information by transmitting the personal identification information to a server of a public institution, the personal identification information being information uniquely defining the applicant, being identification information issued by a public institution, and being acquired at a time of responding to the identity verification request, and also generate the immune certificate, based on the acquired infectious disease response information.

2. The cooperation server according to claim 1, wherein the at least one processor is further configured to execute the instructions to:
transmit the generated immune certificate to a user terminal being a transmission source of a request for generating the immune certificate.

3. The cooperation server according to claim 1, wherein the at least one processor is further configured to execute the instructions to:
generate the immune certificate, based on information on testing of the applicant for an infectious disease or information relating to vaccination of the applicant for an infectious disease.

4. The cooperation server according to claim 1, wherein the at least one processor is further configured to execute the instructions to:
generate the immune certificate including an effective period.

5. The cooperation server according to claim 1, wherein the personal identification information is a personal number.

6. A system comprising:
a user terminal configured to transmit a request for generating an immune certificate for an infectious disease;
an authentication server configured to store information relating to each of a plurality of users whose identities are confirmed; and
a cooperation server being connected to the user terminal and the authentication server, wherein the cooperation server includes
at least one memory configured to store instructions, and
at least one processor configured to execute the instructions to:
receive a request for generating the immune certificate,
transmit, to the authentication server, an identity verification request relating to an applicant who has made a request for generating the immune certificate; and
acquire infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request, and also generate the immune certificate, based on the acquired infectious disease response information.

7. An immune certificate generation method comprising:
receiving a request for generating an immune certificate for an infectious disease;
transmitting an identity verification request relating to an applicant who has made a request for generating the immune certificate, to an authentication server storing information relating to each of a plurality of users whose identities are confirmed;
acquiring infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request; and
generating the immune certificate, based on the acquired infectious disease response information.

8. A non-transitory computer-readable medium that is readable by a computer and stores a program for causing a computer mounted on a cooperation server to execute:
processing of receiving a request for generating an immune certificate for an infectious disease;
processing of transmitting an identity verification request relating to an applicant who has made a request for generating the immune certificate, to an authentication server storing information relating to each of a plurality of users whose identities are confirmed;
processing of acquiring infectious disease response information relating to an applicant of personal identification information, by transmitting the personal identification information to a server of a public institution, the personal identification information being information uniquely defining the applicant, being identification information issued by a public institution, and being acquired at a time of responding to the identity verification request; and
processing of generating the immune certificate, based on the acquired infectious disease response information.
